(19) United States
(12) Patent Application Publication
(10) Pub. No.: US 2015/0241429 A1
(43) Pub. Date: Aug. 27, 2015

(54) IRHOM2 INHIBITION FOR THE TREATMENT OF COMPLEMENT MEDIATED DISORDERS

(71) Applicants: HOSPITAL FOR SPECIAL SURGERY, New York, NY (US); UNIVERSITY HEALTH NETWORK, Toronto (CA)

(72) Inventors: Priya Issuree, New York, NY (US); Thorsten Maretzky, New York, NY (US); David McIlwain, Toronto (CA); Tak Wah Mak, Toronto (CA); Carl Blobel, New York, NY (US); Jane Salmon, New York, NY (US)

(21) Appl. No.: 14/427,185

(22) PCT Filed: Sep. 11, 2013
(86) PCT No.: PCT/US2013/059246

§ 371 Date: Mar. 10, 2015

Related U.S. Application Data
(60) Provisional application No. 61/699,580, filed on Sep. 11, 2012.

(51) Int. Cl. G01N 33/573; C12Q 1/68; C12Q 1/37

(52) U.S. Cl. CPC G01N 33/573; C12Q 1/37; C12Q 1/6876; G01N 2333/96486; G01N 2333/705; G01N 2500/02; G01N 2500/10; G01N 2333/525; C12Q 2600/158; C12Q 2600/136

(57) ABSTRACT

Disclosed are methods for treating a subject with a Complement mediated (e.g., C5a mediated) disease or an immune complex mediated disease. The method comprises the step of administering to the subject an effective amount of an agent that decreases the biological activity of iRhom2 or an agent that modulates formation of a complex between iRhom2 and TACE. Also disclosed are assays for identifying such agents.

### RELATED APPLICATIONS

This application claims the benefit of U.S. Provisional Application No. 61/699,580, filed Sep. 11, 2012, the entire contents of which are hereby incorporated by reference.

### BACKGROUND

Rheumatoid arthritis is a destructive, inflammatory joint disease that affects between 0.5-1% of the adult US population. Studies of mouse models of inflammatory arthritis have yielded mechanistic insights into the pathogenesis of rheumatoid arthritis, and indicate that dysregulation of inflammatory cytokines, immune complexes and anaphylatoxin C5a are principal mediators of rheumatoid arthritis (Firestein, 2003; McInnes and Schett, 2011). Deposits of immunoglobulin and complement are abundant in affected joints of patients with rheumatoid arthritis and in animal models of rheumatoid arthritis in which antibodies are demonstrably pathogenic (Ji et al., 2002a; Ji et al., 2002b). It has also been shown that TNFα is the pivotal element of the inflammatory cytokine network in rheumatoid arthritis synovium. The pathways through which these pathogenic mechanisms interact, and, specifically, whether and how complement and immune complexes drive the production of the pro-inflammatory cytokine TNFα in rheumatoid arthritis remain incompletely defined. Understanding these pathways will be critically important in developing new therapies for rheumatoid arthritis aimed at preventing the production of pathogenic TNF, rather than simply blocking its effects. TNFα is synthesized as a membrane anchored precursor and is released from cells by TNFα convertase (TACE), also referred to as ADAM17 (a disintegrin and metalloproteinase (Black et al., 1997; Horiuchi et al., 2007; Moss et al., 1997).

Inhibitors of TACE are effective in preventing inflammatory arthritis in animal models (Moss, M. L., Sklair-Tavron, L. & Nudelman, R. Drug insight: tumor necrosis factor-converting enzyme as a pharmaceutical target for rheumatoid arthritis. Nat Clin Pract Rheumatol 4, 300-309 (2008)), however clinical trials with a promising TACE inhibitor from Bristol-Meyers Squibb have unfortunately uncovered potential liver toxicity as a serious side effect. The question of whether the liver toxicity encountered in clinical trials was caused by an “on target” toxicity because of inactivation of TACE, or “off target” toxicity because of blocking a different target, was recently addressed through the identification of the first patient lacking TACE (Blaydon, D. C., et al. Inflammatory skin and bowel disease linked to ADAM17 deletion. N Engl J Med 365, 1502-1508 (2011)). This patient suffers from inflammatory skin disease and occasional intestinal inflammation, but is otherwise healthy without any evidence for liver toxicity or other pathologies. The identification of this patient lacking TACE strongly suggests that targeting TACE will not cause liver toxicity, but instead leads to episodes of skin and intestinal inflammation.

As such, there is a need for new agents which can inhibit the pathway mediated by TACE without the skin and intestinal inflammation side effects associated with agents that directly inhibit TACE.

### SUMMARY OF THE INVENTION

Rheumatoid arthritis is triggered by inflammatory mediators such as cytokines, immune complexes and the complement system (including anaphylatoxin C5a), yet much remains to be learned about the underlying mechanisms. It is shown herein that C5a and immunecomplexes activate the principal TNFα convertase, TACE (ADAM17), thereby triggering release of TNFα, and that mice lacking TACE in myeloid cells are protected from inflammatory arthritis in the K/B×N serum transfer model. These results provide the first genetic evidence for a critical role of TACE in myeloid cells in inflammatory arthritis, and suggest that selective inactivation of TACE in myeloid cells should protect from rheumatoid arthritis without causing the skin defects and intestinal inflammation recently described in a TACE-deficient patient. Unexpectedly, it has now been discovered that this effect can be achieved by targeting iRhom2, a novel regulator of TACE. This discovery is based on the findings that iRhom2−/− mice have no active TACE in immune cells and are protected from K/B×N arthritis, but have mature TACE in other tissues, most likely explaining their lack of spontaneous pathological phenotypes. This study identifies the iRhom2/TACE/TNFα signaling axis as a central component of inflammatory arthritis initiated by complement and pathogenic antibodies, and indicates that iRhom2 is an attractive target for selective inactivation of TACE in immune cells without the side effects of systemic inactivation. Based on these discoveries, methods of treating Complement (e.g., C5a) and immune complex mediated disorders such as rheumatoid arthritis by modulating the interaction between iRhom2 and TACE and/or inhibiting the biological activity of iRhom2 are disclosed herein. These methods are expected to be devoid of the side effects associated with systemic inhibition of TACE. Also disclosed are assays for identifying agents that modulate the interaction between iRhom2 and TACE and thereby have potential utility in treating Complement (e.g., C5a) and immune complex mediated diseases.

One embodiment of the invention is a method for treating a subject with a Complement mediated (e.g., C5a mediated) disease or an immune complex mediated disease. The method comprises the step of administering to the subject an effective amount of an agent that decreases the biological activity of iRhom2.

Another embodiment of the invention is a method for treating a subject with a Complement mediated (e.g., C5a mediated) disease or an immune complex mediated disease. The method comprises the step of administering to the subject an effective amount of an agent that modulates formation of a complex between iRhom2 and TACE.

Yet another embodiment of the invention is a method of identifying an agent for the treatment of a disease mediated by Complement (e.g., C5a) or a disease mediated by immune complexes. The method comprises the steps of:
a) combining TACE, iRhom2 and a test agent under conditions suitable for forming a complex between TACE and iRhom2; and
b) assessing the quantity of complex formation between TACE and iRhom2, wherein diminished or increased complex formation between TACE and IRhom2 in the presence of the test agent than in the absence of the test agent is indicative that the test agent is useful for the treatment of a disease mediated by Complement (e.g., C5a) or a disease mediated by immune complexes.

Yet another embodiment of the invention is a method of identifying an agent for the treatment of a disease mediated by Complement (e.g., C5a) or a disease mediated by immune complexes. The method comprises the steps of:
a) combining TNF alpha, Complement (e.g., C5a) or immune complex stimulated myeloid cells in the presence of a test agent under conditions suitable for stimulating TNF alpha release; and
b) assessing the quantity of TNFalpha release, wherein diminished TNFalpha release in the presence of the test agent than in the absence of the test agent is indicative that the test agent is useful for the treatment of a disease mediated by Complement (e.g., C5a) or a disease mediated by immune complexes.

### BRIEF DESCRIPTION OF THE FIGURES

FIG. 1. Complement C5a and Immune Complexes activate TACE. (A,B) ELISA quantification of C5a-induced release of soluble TNFα from bone-marrow-derived macrophages from Tace^{ΔMC} or C5aR−/− mice (A) or fetal liver-derived macrophages from Tace−/− mice (B) following 3-4 hours stimulation compared to controls. 10 ng/ml LPS was used as a positive control, n=4, mean±SEM. Data is representative of three independent experiments (mean±s.d. of samples stimulated in duplicates). * indicates p<0.05.

FIG. 2. Production of TNFα and other pro-inflammatory molecules is enhanced by TNFα shedding. (A) Quantification of mRNA levels of the indicated genes by qPCR following 30 min pre-treatment with 10 μg/ml etanercept and stimulation with C5a (representative of 3 experiments). (B) qPCR of iRhom2 mRNA in human monocytes treated for the indicated time periods with hTNFα (representative of 4 experiments). (C) Relative iRhom2 expression in RA synovial macrophages compared to macrophages from normal control patients, n=7 for control, and n=8 for RA patients (Nonparametric Mann-Whitney U test) (Gordon et al., 2012). (D) iRhom2 expression in synovial macrophages from RA patients following incubation overnight±etanercept, shown as percent of expression in freshly isolated cells, n=8. (Paired sample Wilcoxon signed rank test) (E) Treatment of human monocytes with siRNA against iRhom2 (iR2) reduced iR2 mRNA, but not TACE mRNA in iR2-treated cells as measured by qPCR (top panel), and of mature TACE, but not pro-TACE in a Western blot (lower panel), whereas treatment TACE siRNA reduced both pro- and mature TACE, as detected by Western blot (lower panel, each panel representative of 3 experiments). (F) Decreased TNFα release from C5a, IC and IC+C5a-stimulated cells treated with iR2 siRNA or TACE siRNA compared to untreated controls (representative of 3 independent experiments, mean±s.d. of samples stimulated in duplicates). *: p<0.05.

FIG. 3. Targeted deletion of TACE in myeloid cells reduces TNFα-production in the passive reverse Arthus reaction and protects from inflammatory arthritis initiated by the K/B×N serum transfer model (A) Soluble TNFα in the peritoneal lavage of mice lacking TACE in myeloid cells (Tace^{ΔMC}) and control littermates injected with ova+anti-ova for 3-4 hrs, n=6. (B,C) Tace^{ΔMC} and controls were subjected to the K/B×N serum transfer model for RA, and ankle thickness/swelling (B) and the inflammation score (C) were determined at different time points (please see materials and methods for details). For each mouse, ankle thickness was calculated as the average of measurements of joint thickness of two ankles, n=14 each (Mann Whitney U test, mean±SEM). (D) Histological analysis of representative ankle joints of Tace^{ΔMC} and controls showing representative examples of inflammation (left panels) and cartilage erosion (right panels) eight days after injection with K/B×N serum. (E) Results of blinded histopathological scoring of ankle sections from Tace^{ΔMC} mice and controls to assess inflammation and cartilage erosion, each scored on a scale of 1-4, Tace^{ΔMC} n=14, controls n=14, mean±SEM. The sum of both values was used as an indicator of the severity of inflammatory arthritis for each mouse. * indicates p<0.05.

FIG. 4. iRhom2 is required for stimulated TNF shedding and the development of RA because of its role in TACE maturation in immune cells, but is not essential for TACE maturation in several somatic tissues. (A) Determination of soluble TNFα in the peritoneal lavage of iRhom2−/− mice and their littermate controls subjected to the reverse passive Arthus reaction, n=4. (B,C) Time course of ankle thickness measurements (B) and inflammation score (C) of iRhom2−/− and control littermates injected with K/B×N serum. For each mouse, ankle thickness was calculated as the average of measurements of joint thickness of two ankles (mean±SEM, n=11 for iRhom2−/−; n=14 for wild type, Mann Whitney U test). (D) Histology of representative ankle joints (left panels) and cartilage erosion (right panels) in iRhom2−/− and control mice treated with K/B×N serum. (E) Blinded histopathological scoring of ankle joints from iRhom2−/− or littermate controls for inflammation and cartilage erosion as described above, iRhom2−/− mice=11, controls n=14.

FIG. 5. (A) TACE-mediated cleavage of CD62L on the cell surface of freshly isolated human neutrophils (representative of 2 experiments). Surface expression of CD62L was assessed by flow cytometry following stimulation with 1 μg/mL of C5a or 25 ng/mL PMA for 20 min in the presence or absence of Marimastat (MM). (B) Quantification of total sheep red blood cells phagocytosed by wild type or Tace−/− bone marrow macrophages. Opsonized sheep red blood cells were stained with CFSE and total fluorescence intensity (TFI) was measured by flow cytometry, n=3, mean+/−SEM; * indicates p>0.05. (C) Release of reactive oxygen species by human monocytes when stimulated with C5a in the presence or absence of Marimastat. ROS was quantified by FACS by measuring the mean intensity of the ROS-reactive dye DHR. These results are representative of 4 independent experiments.

FIG. 6. Graphic representation of the response of mutant and control mice to the K/B×N model. The percent of all animals of a specific genotype (Tace^{ΔMC} mice, iRhom2−/− mice and control mice for each mutant) that had low damage (green), moderate damage (yellow) or severe damage (red) as determined by the total clinical score is shown as a bar graph (A) and table (B). These results correspond to a break-down of the data for the analysis at day 8 in FIGS. 3 and 4C.

### DETAILED DESCRIPTION

The “complement system” or simply “Complement” refers to the part of the immune system called the innate immune system that is not adaptable and does not change over the course of an individual's lifetime. However, it can be recruited and brought into action by the adaptive immune system. The complement system helps or “complements” the ability of antibodies and phagocytic cells to clear pathogens from an organism.

The complement system consists of a number of small proteins found in the blood, generally synthesized by the liver, and normally circulating as inactive precursors (pro-proteins). When stimulated by one of several triggers, proteases in the system cleave specific proteins to release cytokines and initiate an amplifying cascade of further cleavages. The end-result of this activation cascade is massive amplification of the response and activation of the cell-killing membrane attack complex. Over 25 proteins and protein fragments make up the complement system, including serum proteins, serosal proteins, and cell membrane receptors. They account for about 5% of the globulin fraction of blood serum.

“Anaphylatoxin C5a”, also referred to herein as simply “C5a”, is a protein fragment released from complement component C5. “Complement component 5” is the fifth component of the complement system, which plays an important role in inflammatory and cell killing processes. In humans, C5a contains 74 amino acids and is composed of four helices and loops connecting the helices. C5a causes release of histamine from mast cells, is an effective leukocyte chemoattractants, causing the accumulation of white blood cells, especially neutrophil granulocytes, at sites of complement activation. C5a is a powerful inflammatory mediator, and seems to be a key factor in the development of pathology of many inflammatory diseases involving the complement system.

“Immune complex” is a complex formed from the integral binding of an antibody to a soluble antigen. The bound antigen acting as a specific epitope, bound to an antibody is referred to as a singular immune complex. After an antigen-antibody reaction, the immune complexes can be subject to any of a number of responses, including complement deposition, opsonization, phagocytosis, or processing by proteases. Immune complexes may themselves cause disease when they are deposited in organs, e.g. in certain forms of vasculitis. This is the third form of hypersensitivity in the Gell-Coombs classification, called Type III hypersensitivity. Immune complex deposition is a prominent feature of several autoimmune diseases, including lupus erythematosus, lupus nephritis, cryoglobulinemia, vasculitis, rheumatoid arthritis, Sjoegren's syndrome, Uveitis, Spondyloarthritis, miscarriage and preeclampsia.

A Complement mediated (e.g., C5a mediated) disease or an immune complex mediated disease is a disease caused, at least in part, by aberrant activity of Complement (e.g., C5a) or by immune complex formation and deposition. Examples include lupus erythematosus, lupus nephritis, cryoglobulinemia, vasculitis, rheumatoid arthritis, Sjoegren's syndrome, Uveitis, Spondyloarthritis, miscarriage and preeclampsia.

Various proteins are described herein by reference to their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

The term “iRhom2”, “Rhbdf2”, or “rhomboid 5 homolog 2 (Drosophila)” refers to a protein having an amino acid sequence substantially identical to any of the representative iRhom2 sequences of GenBank Accession Nos. NP_{—}001005498.2 or NP_{—}078875.4 (human), NP_{—}001161152.1 (mouse) and NP_{—}001100537.1 (rat). Suitable cDNA encoding iRhom2 are provided at GenBank Accession Nos. NM_{—}001005498.3 or NM_{—}024599.5 (human), BC052182.1 (mouse) and NM_{—}001107067.1 (rat).

The term “biological activity of iRhom2” refers to any biological activity associated with the full length native iRhom2 protein, including the biological activity resulting from its associate with TACE. In suitable embodiments, the iRhom2 biological activity is equivalent to the activity of a protein having an amino acid sequence represented by GenBank Accession No. NP_{—}001005498.2, NP_{—}078875.4, NP_{—}001161152.1, or NP_{—}001100537.1. Decreasing the biological activity, in one embodiment, refers to decreasing the expression of the iRhom2 mRNA or protein. Measurement of transcriptional activity can be performed using any known method, such as immunohistochemistry, reporter assay or RT-PCR, which can also be used to determine whether the biological activity of iRhom2 is decreased. The iRhom2 referred to herein can be a mammalian iRhom2 or in a particular aspect, a human iRhom2.

The term “TNF” or “tumor necrosis factor” refers to a cytokine family that can cause cell death (apoptosis). Representative factors in this family are tumor necrosis factor-alpha (TNF-α) and tumor necrosis factor-beta (TNF-β).

The term “TNF-α” or “tumor necrosis factor-alpha” refers to a protein having an amino acid sequence substantially identical to any of the representative TNF-α sequences of GenBank Accession Nos. NP_{—}000585.2 (human), NP_{—}038721.1 (mouse) and NP_{—}036807.1 (rat). Suitable cDNA encoding TNF-α are provided at GenBank Accession Nos. NM_{—}000594.2 (human), NM_{—}013693.2 (mouse) and NM_{—}012675.3 (rat).

TNF is primarily produced as a transmembrane protein. From this membrane-integrated form the soluble homotrimeric cytokine, the “soluble TNF” (sTNF) is released via proteolytic cleavage by the metalloprotease TNF alpha converting enzyme (TACE, also called ADAM17).

Production of soluble TNF by a cell can be measured by the amount of sTNF released from the cell membrane with methods known in the art, such as ELISA, FACS, Western blot and immunohistochemistry. When compared to a suitable control, increased or decreased production of sTNF can be used to determine whether an agent inhibits or increases the production of sTNF.

The term “TACE”, “ADAM17” or “ADAM metallopeptidase domain 17” refers to a protein having an amino acid sequence substantially identical to any of the representative TACE sequences of GenBank Accession Nos. NP_{—}003174.3 (human), NP_{—}033745.4 (mouse) and NP_{—}064702.1 (rat). Suitable cDNA encoding TACE are provided at GenBank Accession Nos. NM_{—}003183.4 (human), NM_{—}009615.5 (mouse) and NM_{—}020306.1 (rat).

TACE and iRhom2 bind together to form a complex and can immunoprecipitate (Adrain et al., Science 335.225 (2012). The ability of an agent to modulate (increase or decrease) binding between TACE and iRhom2 is disclosed herein to correlate with the ability of the agent to modulate the activity of iRhom2, and by extension, TACE. The amount of complex formation can be measured by methods known in the art (see Adrian et al., supra), and include immunoprecipitation with tagged iRhom2 or tagged TACE. For example, the binding partners can be expressed in eukaryotic cell expression systems, and tested for antibodies or reagents that prevent binding, dissociate bound molecules, or stabilize the interaction, with, for example, pulldown assays, assays where one binding partner is immobilized on a plate and the second one is tagged and added. The quantity of the tagged molecule released into the supernatant can then be assessed by measuring the amount of released tagged protein by Western blot, dot blot or ELISA. An enzyme tag can be used, such as alkaline phosphatase, in which case the release can be measure by colorimetric determination of alkaline phosphatase activity in the supernatant A fluorescent protein tag can be added, in which case the release can be measure by a fluorimeter.

A “biological equivalent” of a protein or nucleic acid refers to a protein or nucleic acid that is substantially identical to the protein or nucleic acid. As used herein, the term “substantially identical”, when referring to a protein or polypeptide, is meant one that has at least 80%, 85%, 90%, 95%, or 99% sequence identity to a reference amino acid sequence. The length of comparison is preferably the full length of the polypeptide or protein, but is generally at least 10, 15, 20, 25, 30, 40, 50, 60, 80, or 100 or more contiguous amino acids. A “substantially identical” nucleic acid is one that has at least 80%, 85%, 90%, 95%, or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

In one aspect of any of the above methods, the agent that decreases the biological activity of iRhom2 is an antibody or antibody fragment that specifically recognizes iRhom2 and inhibits the activity of TACE; a small molecule inhibitor of iRhom2; a polypeptide decoy mimicking a domain necessary for the interaction of TACE and iRhom2; a miRNA, a siRNA, a shRNA, a dsRNA or an antisense RNA directed to iRhom2 DNA or mRNA; a polynucleotide encoding the miRNA, siRNA, shRNA, dsRNA or antisense RNA; or an equivalent of each thereof.

In one aspect, the agent that decreases the biological activity of iRhom2 is an antibody or antibody fragment that specifically recognizes iRhom2 and inhibits the activity of TACE, or a polypeptide decoy mimicking a domain necessary for the interaction of TACE and iRhom2. In a particular aspect, the antibody or antibody fragment specifically recognizes an extracellular domain of iRhom2. For example, the antibody or antibody fragment recognizes and specifically binds to the polypeptide GPSDKSDLSQKQPSAVVCHQDPRTCEEPASSGAHIWPDDITKWPICTEQAQS (SEQ ID NO 1) or an antibody binding fragment thereof containing 5 to 10, 10 to 15, 15 to 20, 20-25, 25-30, 30-40, 40-45 or more than 45 amino acids. Alternatively, the antibody or antibody fragment recognizes and specifically binds to a transmembrane region of iRohm2 or a region that includes both the extracellular loop and the transmembrane region. In another aspect, the agent further comprises a cell penetrating peptide. The cell penetrating peptide, in one aspect, comprises a HIV-TAT peptide.

Agents which modulate the formation of a complex between iRhom2 and TACE include compounds that increase (e.g., stabilize) or decrease (e.g., destabilize or inhibit) the binding between the two proteins, resulting in more complex formation or less complex formation, respectively. Examples of agents that inhibit binding include an antibody or an antibody fragment that specifically recognizes the iRhom2 protein, and preferably the extracellular loop of either iRhom2 (the polypeptide GPSDKSDLSQKQPSAVVCHQDPRTCEEPASSGAHIWPDDITKWPICTEQAQS (SEQ ID NO 1) or an antibody binding fragment thereof containing 5 to 10, 10 to 15, 15 to 20, 20-25, 25-30, 30-40, 40-45 or more than 45 amino acids. Alternatively, the antibody or antibody fragment that specifically recognizes a transmembrane domain of iRohm2 or a region comprising the extracellular domain and a transmembrane domain. In another alternative, the agent that inhibits binding is an antibody or an antibody fragment that specifically recognizes the extracellular domain of either TACE (the polypeptide murine TACE accession number: http://www.ncbi.nlm.nih.gov/protein/NP_{—}033745.4—the extracellular domain is between aa #1 and ˜670; and human TACE accession number: http://www.ncbi.nlm.nih.gov/protein/NP_{—}003174.3—the extracellular domain is between aa #1 and ˜670), SEQ ID NO 1) or an antibody binding fragment thereof containing 5 to 10, 10 to 15, 15 to 20, 20-25, 25-30, 30-40, 40-45 or more than 45 amino acids. In another alternative, the inhibitor of complex formation can be a small molecule which binds either iRhom2 or TACE in the region where the two proteins bind, e.g., a fragment of either protein which binds the other or a decoy that mimics a domain necessary for the interaction of TACE and iRhom2. This region can also include the transmembrane domain of TACE and one or more of the seven transmembrane domains of iRhom2. Agents which inhibit the formation of a complex between iRohm2 and TACE also include compounds which suppress the expression of iRohm2, e.g., iRNA can be a miRNA, a siRNA, a shRNA, a dsRNA or an antisense RNA directed to iRhom2 DNA or mRNA, or a polynucleotide encoding the miRNA, siRNA, shRNA, dsRNA or antisense RNA, a vector comprising the polynucleotide Agents that increase complex formation include antibodies or antibody fragments or small molecules that bind to and stabilize the complex. The would be identified from combinatorial chemistry inhibitor libraries by screens, and then further optimized through chemical alterations. In another aspect, the agent further comprises a cell penetrating peptide. The cell penetrating peptide, in one aspect, comprises a HIV-TAT peptide.

“Short interfering RNAs” (siRNA) refer to double-stranded RNA molecules (dsRNA), generally, from about 10 to about 30 nucleotides in length that are capable of mediating RNA interference (RNAi). “RNA interference” (RNAi) refers to sequence-specific or gene specific suppression of gene expression (protein synthesis) that is mediated by short interfering RNA (siRNA). As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA. A siRNA may be chemically modified to increase its stability and safety. See, e.g. Dykxhoorn and Lieberman (2006) Annu. Rev. Biomed. Eng. 8:377-402 and U.S. Patent Application Publication No.: 2008/0249055.

“Double stranded RNAs” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

“MicroRNAs” (miRNA) refer to single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

siRNA, dsRNA, and miRNA to inhibit gene expression can be designed following procedures known in the art. See, e.g., Dykxhoorn and Lieberman (2006) Annu. Rev. Biomed. Eng. 8:377-402; Dykxhoorn et al. (2006) Gene Therapy 13:541-52; Aagaard and Rossi (2007) Adv. Drug Delivery Rev. 59:75-86; de Fougerolles et al. (2007) Nature Reviews Drug Discovery 6:443-53; Krueger et al. (2007) Oligonucleotides 17:237-250; U.S. Patent Application Publication No.: 2008/0188430; and U.S. Patent Application Publication No.: 2008/0249055.

Delivery of siRNA, dsRNA or miRNA to a cell can be made with methods known in the art. See, e.g., Dykxhoorn and Lieberman (2006) Annu. Rev. Biomed. Eng. 8:377-402; Dykxhoorn et al. (2006) Gene Therapy 13:541-52; Aagaard and Rossi (2007) Adv. Drug Delivery Rev. 59:75-86; de Fougerolles et al. (2007) Nature Reviews Drug Discovery 6:443-53; Krueger et al. (2007) Oligonucleotides 17:237-250; U.S. Patent Application Publication No.: 2008/0188430; and U.S. Patent Application Publication No.: 2008/0249055.

“Antisense” oligonucleotides have nucleotide sequences complementary to the protein coding or “sense” sequence. Antisense RNA sequences function as regulators of gene expression by hybridizing to complementary mRNA sequences and arresting translation (Mizuno et al. (1984) PNAS 81:1966; Heywood et al. (1986) Nucleic Acids Res. 14:6771). An antisense polynucleotide comprising the entire sequence of the target transcript or any part thereof can be synthesized with methods known in the art. See e.g., Ferretti et al. (1986) PNAS 83:599. The antisense polynucleotide can be placed into vector constructs, and effectively introduced into cells to inhibit gene expression (Izant et al. (1984) Cell 36:1007). Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the gene is retained as a functional property of the polynucleotide.

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provide desirable properties (e.g., increased nuclease-resistance, tighter-binding, stability or a desired Tₘ). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al. (1991) Science 254:1497) or incorporating 2′-O-methyl ribonucleotides, phosphorothioate nucleotides, methyl phosphonate nucleotides, phosphotriester nucleotides, phosphorothioate nucleotides, phosphoramidates. Another example of the modification is replacement of a non-bridging phosphoryl oxygen atom with a sulfur atom which increases resistance to nuclease digestion. Increased antisense polynucleotide stability can also be achieved using molecules with 2-methyoxyethyl substituted backbones. See e.g., U.S. Pat. Nos. 6,451,991 and 6,900,187.

In another embodiment, ribozymes can be used (see, e.g., Cech (1995) Biotechnology 13:323; and Edgington (1992) Biotechnology 10:256 and Hu et al., PCT Publication WO 94/03596). A ribonucleic acid enzyme (“ribozymes”, “RNA enzyme”, or “catalytic RNA”) is an RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. Methods of making and using ribozymes can be found in e.g., U.S. Patent Application Publication No. 2006/0178326.

“Triplex ribozymes” configurations allow for increased target cleavage relative to conventionally expressed ribozymes. Examples of triplex ribozymes include hairpin ribozymes and hammerhead ribozymes. Methods of making and using triplex ribozymes are found in, e.g., Aguino-Jarguin et al. (2008) Oligonucleotides 18(3):213-24 and U.S. Patent Application Publication No. 2005/0260163.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz (1996) Current Opinion in Neurobiology 6:629-634. Another subsequence, the h (hydrophobic) domain of signal peptides, was found to have similar cell membrane translocation characteristics (see, e.g., Lin et al. (1995) J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

The present disclosure provides, in one embodiment, a polypeptide decoy that mimics a domain necessary for the interaction of TACE and iRhom2 for decreasing the biological activity of iRhom2. A polypeptide decoy of a protein for inhibiting the interaction between the protein and a second protein is a polypeptide that binds to the second protein but does not carry out the biological activity that such a binding would normally carry out.

In one embodiment, a polypeptide decoy is a fragment of the iRhom2 protein that includes the iRhom2 extracellular domain responsible for binding TACE, e.g., a polypeptide with the amino sequence of SEQ ID NO 1 or a 5 to 10, 10 to 15, 15 to 20, 20-25, 25-30, 30-40, 40-45 or more than 45 amino acid fragment thereof that binds TACE. In another embodiment, the polypeptide decoy does not include an iRhom2 domain that is responsible for activating TACE or contains a mutation at this domain so that the polypeptide decoy does not activate TACE. Alternatively, the polypeptide decoy also includes a portion of the transmembrane domain of iRhom2, together with or in the absence of the extracellular domain.

In another embodiment, a polypeptide decoy is a fragment of the TACE protein that includes the TACE extracellular domain responsible for binding iRhom2, e.g., or a 5 to 10, 10 to 15, 15 to 20, 20-25, 25-30, 30-40, 40-45 or more than 45 amino acid fragment thereof that binds with iRohm2. In another embodiment, the polypeptide decoy does not include an TACE domain that is responsible for its shedding activity or contains a mutation at this domain so that the polypeptide decoy does not have shedding activity. Alternatively, the polypeptide decoy also includes a portion of the transmembrane domain of TACE together with or in the absence of a portion of the extracellular domain of TACE.

“Antibody” is intended to encompass both polyclonal and monoclonal antibodies. The terms polyclonal and monoclonal refer to the degree of homogeneity of an antibody preparation, and are not intended to be limited to particular methods of production. “Antibody” also encompasses functional fragments of antibodies, including fragments of chimeric, humanized, primatized, veneered or single chain antibodies. For example, an antibody can be an IgG or antigen-binding fragment of an IgG. Antibody fragments include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain fragment can be designed to include DNA sequences encoding the CH₁ domain and hinge region of the heavy chain.

Single chain antibodies, and chimeric, humanized or primatized (CDR-grafted), or veneered antibodies, as well as chimeric, CDR-grafted or veneered single chain antibodies, comprising fragments derived from different species, and the like are also encompassed by the term “antibody”. The various fragments of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for a mammalian (e.g., human) specific portion of iRohm2 and TACE that affect binding between the two proteins or which inhibit a biological activity of iRohm2 can be raised against an appropriate immunogen, such as isolated and/or recombinant extracellular loop of iRohm2 or the extracellular domain of TACE, with or without the transmembrane domains attached, or fragments thereof (including synthetic molecules, such as synthetic peptides).

Preparation of immunizing antigen, and polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977), Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Generally, a hybridoma is produced by fusing a suitable immortal cell line (e.g., a myeloma cell line such as SP2/0, P3X63Ag8.653 or a heteromyloma) with antibody producing cells. Antibody producing cells can be obtained from the peripheral blood or, preferably the spleen or lymph nodes, of humans or other suitable animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity (e.g., human antibodies or antigen-binding fragments) can be used, including, for example, methods which select recombinant antibody from a library (e.g., a phage display library), or which rely upon immunization of transgenic animals (e.g., mice) capable of producing a repertoire of human antibodies (see e.g., Jakobovits et al., Proc. Natl. Acad. Sci. USA, 90: 2551-2555 (1993); Jalkobovits et al., Nature, 362: 255-258 (1993); Lonberg et al., U.S. Pat. No. 5,545,806; Surani et al., U.S. Pat. No. 5,545,807; Lonberg et al., WO97/13852).

In one embodiment, the antibody or antigen-binding fragment used in the disclosed methods binds to a fragment of the extracellular loop of iRohm2 or TACE. The fragment can be 5 to 10 amino acids long, 10 to 15 amino acids long, 15 to 20 amino acids long, 20-25 amino acids long, 25-30 amino acids long, 30-35 amino acids long, 35-40 amino acids long, 40-45 amino acids long or greater than 45 amino acids long.

The compositions described herein for a therapeutic use may be administered with an acceptable pharmaceutical carrier. Acceptable “pharmaceutical carriers” are well known to those of skill in the art and can include, but not be limited to any of the standard pharmaceutical carriers, such as phosphate buffered saline, water and emulsions, such as oil/water emulsions and various types of wetting agents.

The term “treating” is meant administering a pharmaceutical composition for the purpose of therapeutic treatment by reducing, alleviating or reversing at least one adverse effect or symptom; or for the purpose of prophylactic treatment. “Prophylactic treatment” means identifying a subject (i.e., a patient) having an increased susceptibility to a disease but not yet exhibiting symptoms of the disease, and administering a therapy according to the principles of this disclosure. The prophylactic therapy is designed to reduce the likelihood that the susceptible subject will later become symptomatic or that the disease will be delayed in onset or progress more slowly than it would in the absence of the preventive therapy. A subject may be identified as having an increased likelihood of developing the disease by any appropriate method including, for example, by identifying a family history of the disease or other TNF-dependent autoimmune disorder, or having one or more diagnostic markers indicative of disease or susceptibility to disease.

The term “administering” for in vivo and ex vivo purposes means providing the subject with an effective amount of the nucleic acid molecule or polypeptide effective to prevent or inhibit a disease or condition in the subject. Methods of administering pharmaceutical compositions are well known to those of skill in the art and include, but are not limited to, microinjection, intravenous or parenteral administration. The compositions are intended for systemic, topical, oral, or local administration as well as intravenously, subcutaneously, or intramuscularly. Administration can be effected continuously or intermittently throughout the course of treatment. Methods of determining the most effective means and dosage of administration are well known to those of skill in the art and will vary with the vector used for therapy, the polypeptide or protein used for therapy, the purpose of the therapy, the target cell being treated, and the subject being treated. Single or multiple administrations can be carried out with the dose level and pattern being selected by the treating physician. For example, the compositions can be administered prior to a subject already suffering from a disease or condition that is linked to apoptosis.

The term “effective amount” refers to a quantity of compound (e.g., an agent that decreases the biological activity of iRhom2 or that inhibits formation of a complex between iRhom2 and TACE) delivered with sufficient frequency to provide a medical benefit to the patient. In one embodiment, an effective amount of a protein is an amount sufficient to treat or ameliorate a symptom of a Complement mediated (e.g., C5a mediated) disease or an immune complex mediated disease. Exemplary effective amounts of agent that decreases the biological activity of iRhom2 or that inhibits formation of a complex between iRhom2 and TACE range from 0.1 ug/kg body weight to 100 mg/kg body weight; alternatively 1.0 ug/kg body weight to 10 mg/kg body weight

A “subject” includes mammals, e.g., humans, companion animals (e.g., dogs, cats, birds and the like), farm animals (e.g., cows, sheep, pigs, horses, fowl and the like) and laboratory animals (e.g., rats, mice, guinea pigs and the like). In a preferred embodiment of the disclosed methods, the subject is human.

The invention also includes a method of identifying an agent for the treatment of a disease mediated by Complement (e.g., C5a) or a disease mediated by immune complexes. The method assesses the ability of a test agent ability of a test agent to modulate (increase or decrease) complex formation between iRohm2 and TACE. The method comprises the step of combining TACE, iRhom2 and a test agent under conditions suitable for forming a complex between TACE and iRhom2. This could be a pre-existing complex of iRhom2 and TACE that is immunoprecipitated from cells, such as myeloid cells. It could also be a complex of recombinantly expression extracellular loop of iRhom2 and extracellular domain of TACE, with tags added, as described above. The amount of complex formation is compared to the amount of complex formed under identical conditions in the absence of the test agent. A greater or lesser amount of complex formation in the presence of the test agent than in its absence is indicative that test agent is effective for the treatment of a disease mediated by Complement (e.g., C5a) or a disease mediated by immune complexes. The efficacy a test agent showing the ability to modulate complex formation between iRohm2 and TACE can be further tested and/or confirmed in additional assays for assessing efficacy against any one or more disease mediated by Complement (e.g., C5a) or a disease mediated by immune complexes. Typically, a plurality of test agents are tested, for example as in high throughput screening, for their ability to modulate complex formation between iRohm2 and TACE. Those test agents demonstrating an ability to modulate complex formation between iRohm2 and TACE are typically selected for further testing in assays for assessing efficacy against any one or more disease mediated by Complement (e.g., C5a) or a disease mediated by immune complexes.

An alternative method for identifying an agent for the treatment of a disease mediated by Complement (e.g., C5a) or a disease mediated by immune complexes assesses the ability of a test agent to inhibit TNF alpha release from Complement (e.g., C5a) or immune complex stimulated myeloid, cells. The method comprises combining Complement (e.g., C5a) or immune complex stimulated myeloid cells in the presence of a test agent under conditions suitable for stimulating TNF alpha release. Exemplary conditions are described in the Experimental. The quantity of TNFalpha release is measured and compared with the quantity released under identical conditions in the absence of the test agent. Diminished TNFalpha release in the presence of the test agents than in its absence is indicative if a test agent useful for the treatment of a disease mediated by Complement (e.g., C5a) or a disease mediated by immune complexes. The efficacy a test agent showing the ability to inhibit TNF alpha release from Complement (e.g., C5a) stimulated or immune complex stimulated myeloid cells can be further tested and/or confirmed in additional assays for assessing efficacy against any one or more disease mediated by Complement (e.g., C5a) or a disease mediated by immune complexes. Typically, a plurality of test agents are tested, for example as in high throughput screening, for their ability to inhibit TNF alpha release from Complement (e.g., C5a) stimulated or immune complex stimulated myeloid cells. Those test agents demonstrating an ability to inhibit TNF alpha release from Complement (e.g., C5a) stimulated or immune complex stimulated myeloid cells complex are typically selected for further testing in assays for assessing efficacy against any one or more disease mediated by Complement (e.g., C5a) or a disease mediated by immune complexes.

Assays for assessing efficacy of a test agent against one or more diseases mediated by Complement (e.g., C5a) or immune complexes include those described in the following paragraphs.

Animals model for assessing efficacy against rheumatoid arthritis are disclosed in Ji et al., 2002a; Ji et al., 2002b); Ji, H., Pettit, A., Ohmura, K., Ortiz-Lopez, A., Duchatelle, V., Degott, C., Gravallese, E., Mathis, D., and Benoist, C. 2002. Critical roles for interleukin 1 and tumor necrosis factor alpha in antibody-induced arthritis. J Exp Med 196:77-85; Kitaura, H., Sands, M. S., Aya, K., Zhou, P., Hirayama, T., Uthgenannt, B., Wei, S., Takeshita, S., Novack, D. V., Silva, M. J., et al. 2004. Marrow stromal cells and osteoclast precursors differentially contribute to TNF-alpha-induced osteoclastogenesis in vivo. J Immunol 173:4838-4846.

Animals model for assessing efficacy against uveitis are disclosed in de Kozak Y, Omri B, Smith J R, Naud M C, Thillaye-Goldenberg B, Crisanti P., Protein kinase Czeta (PKCzeta) regulates ocular inflammation and apoptosis in endotoxin-induced uveitis (EIU): signaling molecules involved in EIU resolution by PKCzeta inhibitor and interleukin-13, Am J Pathol. 2007 April; 170(4):1241-57; Calder C J, Nicholson L B, Dick A D, J Immunol., A selective role for the TNF p55 receptor in autocrine signaling following IFN-gamma stimulation in experimental autoimmune uveoretinitis, 2005 Nov. 15; 175(10):6286-93; Invest Ophthalmol Vis Sci., Brito B E, O'Rourke L M, Pan Y, Anglin J, Planck S R, Rosenbaum J T, IL-1 and TNF receptor-deficient mice show decreased inflammation in an immune complex model of uveitis 1999 October; 40(11):2583-9.

Assays for assessing efficacy against lupus are described in Venegas-Pont, M., et al. Tumor necrosis factor-alpha antagonist etanercept decreases blood pressure and protects the kidney in a mouse model of systemic lupus erythematosus. Hypertension 56, 643-649 (2010); Aringer, M. & Smolen, J. S. The role of tumor necrosis factor-alpha in systemic lupus erythematosus. Arthritis Res Ther 10, 202 (2008); Yokoyama, H., Kreft, B. & Kelley, V. R. Biphasic increase in circulating and renal TNF-alpha in MRL-Ipr mice with differing regulatory mechanisms. Kidney Int 47, 122-130 (1995); Herrera-Esparza, R., Barbosa-Cisneros, O., Villalobos-Hurtado, R. & Avalos-Diaz, E. Renal expression of IL-6 and TNFalpha genes in lupus nephritis. Lupus 7, 154-158 (1998); Brennan, D. C., Yui, M. A., Wuthrich, R. P. & Kelley, V. E. Tumor necrosis factor and IL-1 in New Zealand Black/White mice. Enhanced gene expression and acceleration of renal injury. J Immunol 143, 3470-3475 (1989); Deng G M, Liu L, Kyttaris V C, Tsokos G C. Lupus serum IgG induces skin inflammation through the TNFR1 signaling pathway. J Immunol. 2010 Jun. 15; 184(12):7154-61. Epub 2010 May 14; Bethunaickan R, Sahu R, Liu Z, Tang Y T, Huang W, Edegbe O, Tao H, Ramanujam M, Madaio M P, Davidson A. Anti-TNF treatment of IFN induced lupus nephritis reduces the renal macrophage response but does not alter glomerular immune complex formation Arthritis Rheum. 2012 Jun. 5. doi: 10.1002/art.34553. [Epub ahead of print].

Assays for assessing efficacy against preeclampsia are described in: Autoantibody-mediated angiotensin receptor activation contributes to preeclampsia through tumor necrosis factor-alpha signaling. Irani R A, Zhang Y, Zhou C C, Blackwell S C, Hicks M J, Ramin S M, Kellems R E, Xia Y. Hypertension. 2010 May; 55(5):1246-53. Epub 2010 Mar. 29; Qing X, Redecha P B, Burmeister M A, Tomlinson S, D'Agati V D, Davisson R L, Salmon J E. Targeted inhibition of complement activation prevents features of preeclampsia in mice. Kidney Intl 79:331-9, 2011. (ePub 13 Oct. 2010).

Assays for assessing efficacy against miscarriage are described in: Berman J, Girardi G, Salmon J E. TNF-alpha is a critical effector and a target for therapy in antiphospholipid antibody-induced pregnancy loss; J Immunol. 2005 Jan. 1; 174(1):485-90. Qing X, Redecha P B, Burmeister M A, Tomlinson S, D'Agati V D, Davisson R L, Salmon J E. Targeted inhibition of complement activation prevents features of preeclampsia in mice. Kidney Intl 79:331-9, 2011. (ePub 13 Oct. 2010).

The invention is illustrated by the following examples, which are not intended to be limiting in any way.

### EXPERIMENTAL

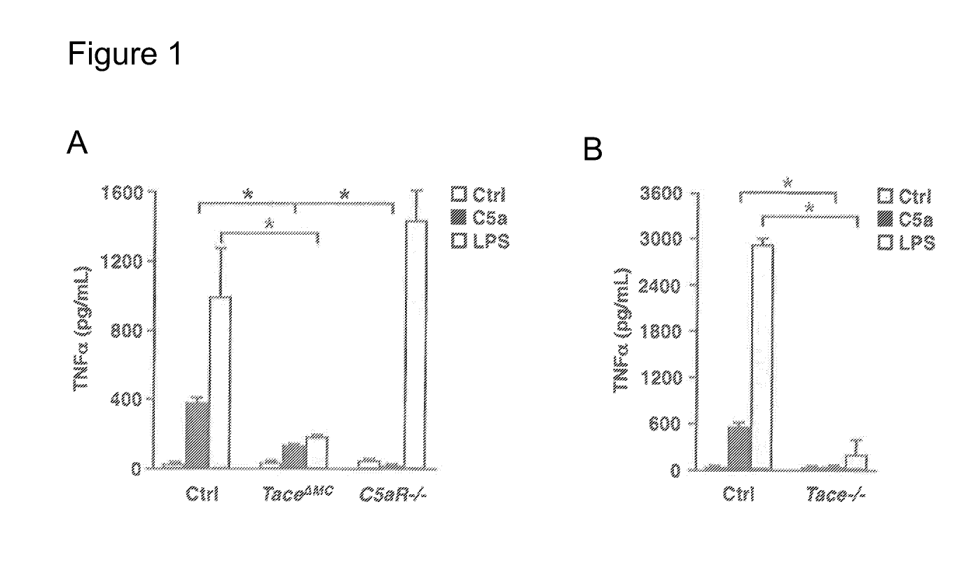

To corroborate that TACE is required for C5a-induced TNFα shedding, we stimulated macrophages from mice lacking TACE in myeloid cells (Tace^{ΔMC}, FIG. 1A) and Tace−/− fetal liver macrophages (FIG. 1B) with C5a and found markedly reduced release of TNFα compared to controls. There was also no TNFα released from C5aR-deficient macrophages treated with C5a (FIG. 1A), demonstrating that C5aR is the relevant receptor stimulated by C5a, and ruling out contamination with LPS. Taken together, our results show that C5a-C5aR interactions activate TACE.

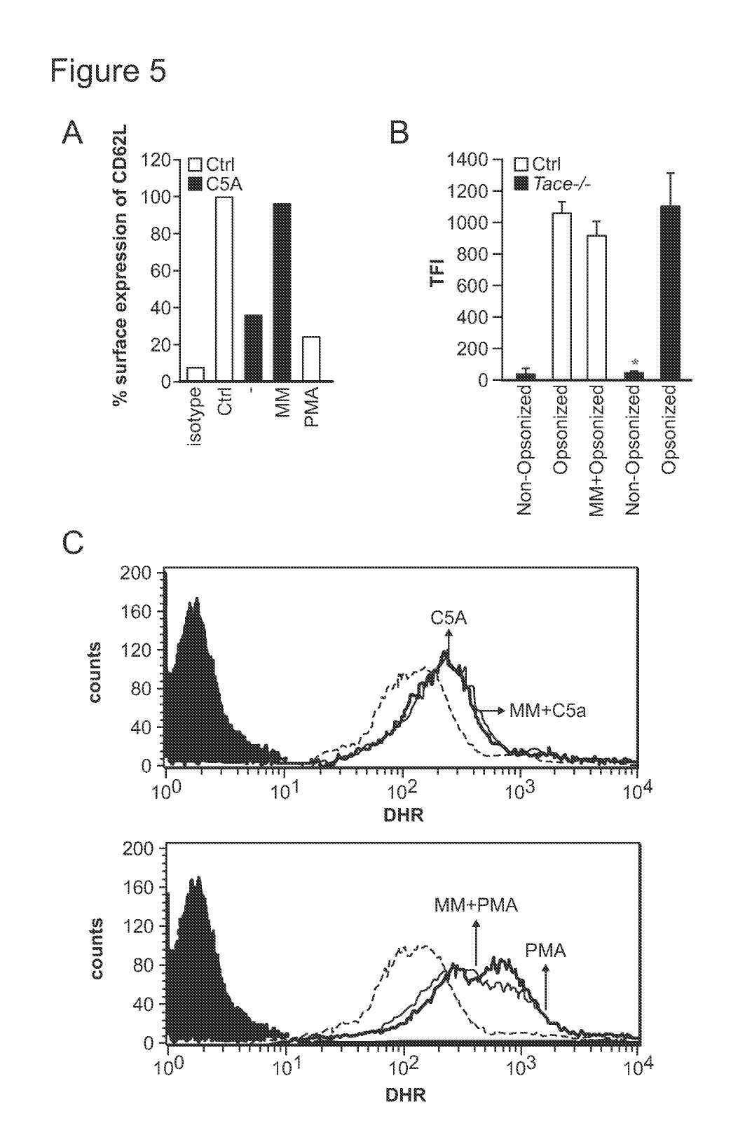

The Fc domains of pathogenic IgG initiate tissue damage by binding FcγR on effector cells and by activating complement, and FcγRs are critical for the pathogenesis of inflammatory arthritis in the K/B×N serum model (Ji et al., 2002a). We also found that Tace−/− fetal liver macrophages internalized IgG-opsonized red blood cells as well as wild type controls, and that inhibition of TACE with MM did not alter opsonization (FIG. 5) or a C5a-stimulated oxidative burst in human monocytes (FIG. 5). These results indicate that TACE is required for C5aR- and FcγR-mediated release of soluble TNFα from leukocytes, but not other C5aR- and FcγR-mediated effector functions essential for microbial host defense, such as phagocytosis and generation of reactive oxygen intermediates.

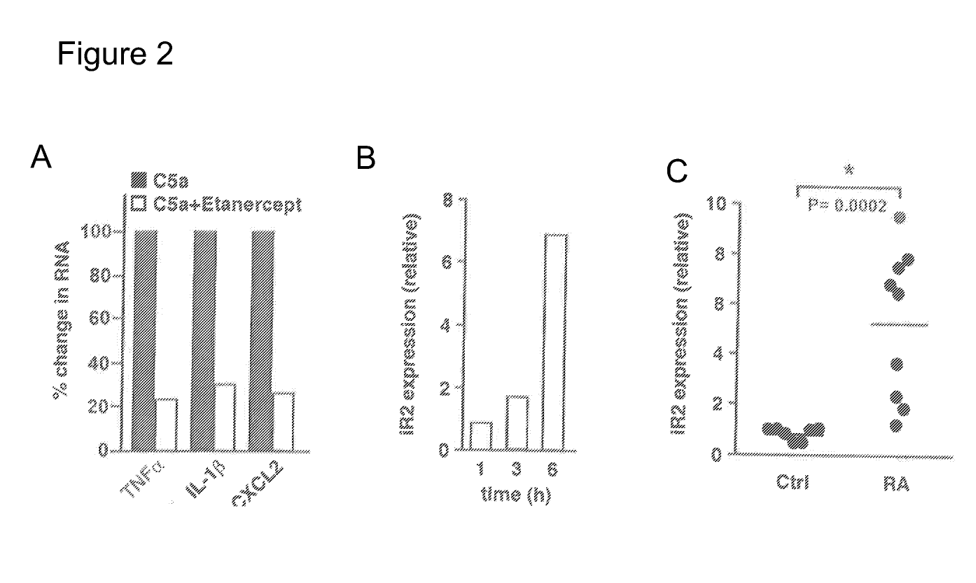

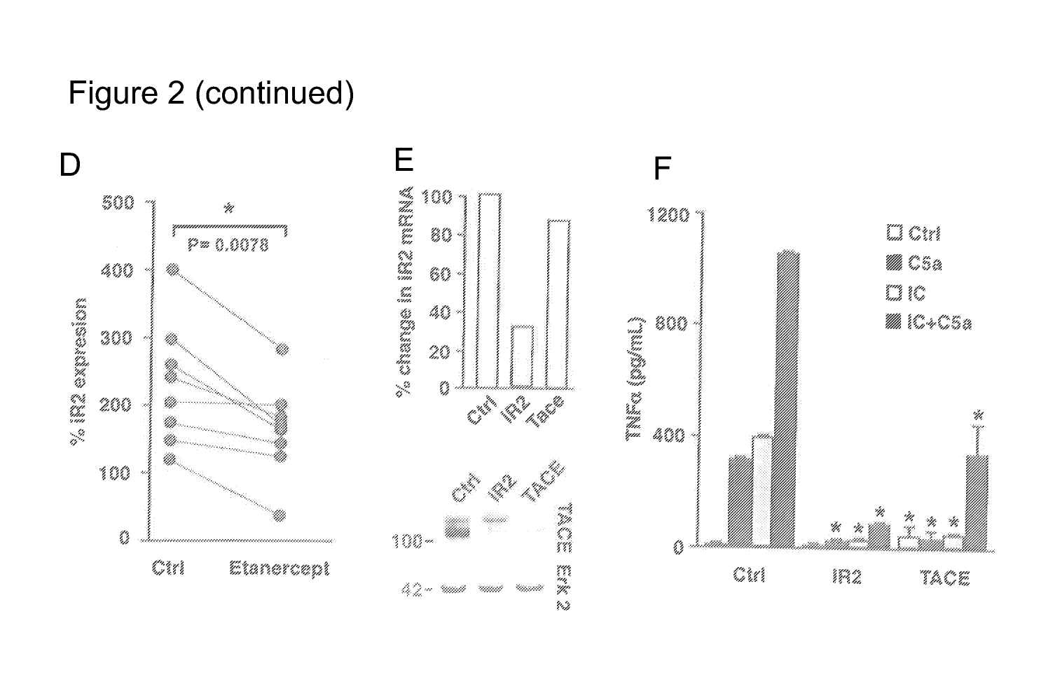

Studies of TNFα mRNA levels in isolated macrophages stimulated with C5a showed that blocking TACE not only abolished the release of soluble TNFα, but also reduced TNFα mRNA by 50%, and significantly reduced the mRNA levels of two other TNFα-responsive genes that are implicated in RA, IL-1β and CXCL2 (Ha et al., 2010; Ohmura et al., 2005). The anti-TNFα biologic drug etanercept had a similar effect on the mRNA levels of these three molecules (FIG. 2A). Interestingly, we also found that TNFα stimulation of human monocytes enhanced the expression of iRhom2, a catalytically inactive member of the seven-membrane spanning Rhomboid family of intramembrane proteinases that has recently emerged as an essential regulator of TACE in myeloid cells (Adrain et al., 2012; McIlwain et al., 2012; Siggs et al., 2012) (FIG. 2B). Expression of iRhom2 was also elevated in freshly isolated synovial macrophages from RA patients (FIG. 2C), raising the possibility that it has a role in the pathogenesis of RA. iRhom2 expression was substantially reduced when etanercept was added to ex vivo cultures of RA synovial macrophages (FIG. 2D), suggesting that elevated expression of iRhom2 in RA synovial macrophages is dependent on TNFα. Together, these findings suggest that inhibitors of TACE could have previously unanticipated anti-inflammatory benefits by reducing not only the shedding of TNFα, but also its biosynthesis (see also (Bashir et al., 2009; Guergnon et al., 2003)). This, in turn, would also reduce the production of TNFα-dependent cytokines and of iRhom2, a crucial positive regulator of TACE (Adrain et al., 2012; McIlwain et al., 2012; Siggs et al., 2012).

To directly test whether iRhom2 plays a role in C5a- or IC-stimulated release of TNFα, we treated human monocytes with siRNA against iRhom2 or TACE. qPCR confirmed that the siRNA against iRhom2 reduced iRhom2 mRNA levels by 70% (FIG. 2E). Moreover, a Western blot analysis showed that iRhom2-siRNA reduced expression of mature, but not the pro-form of TACE, as previously described in iRhom2−/− mouse myeloid cells (Adrain et al., 2012; McIlwain et al., 2012; Siggs et al., 2012), whereas siRNA against TACE reduced the expression of both forms (FIG. 2E). Treatment with siRNA against iRhom2 or TACE significantly decreased TNFα release following stimulation by C5a, IC or both, establishing that iRhom2 and TACE are key molecules in the complement- and immune complex-stimulated TNFα release from human monocytes (FIG. 2F).

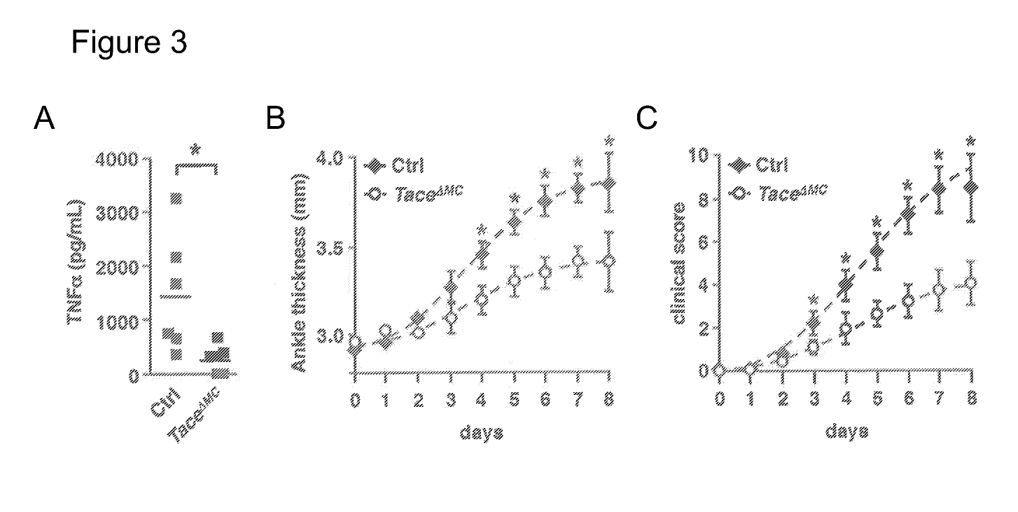

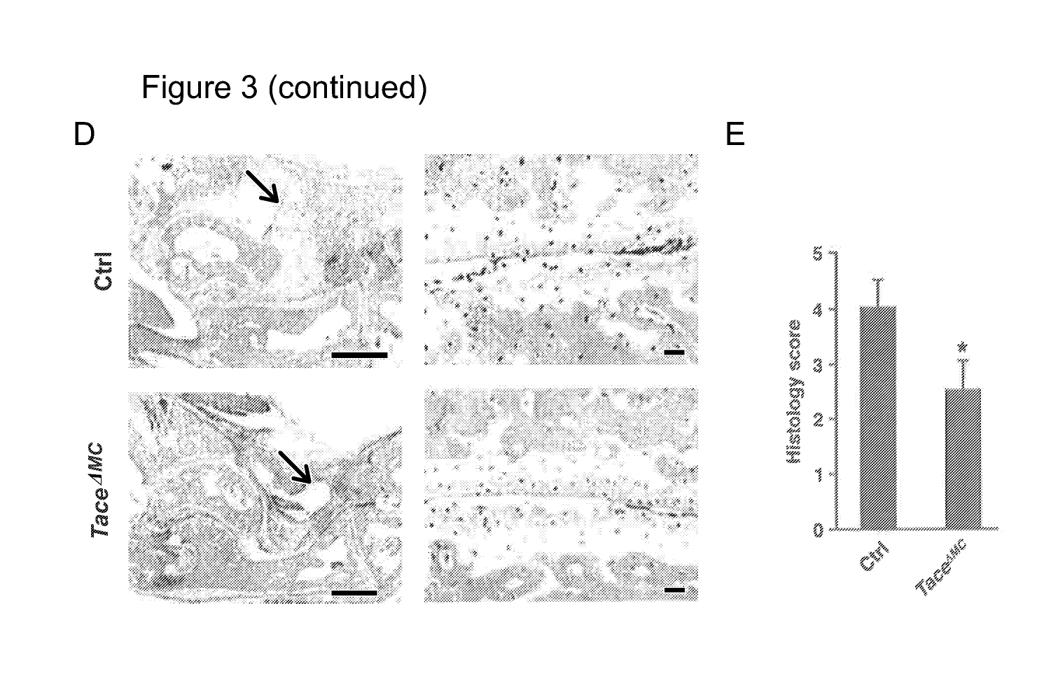

To assess the importance of C5a- and FcγR-stimulated activation of TACE in vivo, Tace^{ΔMC} mice were subjected to the reverse passive Arthus reaction of peritonitis, in which immune complex deposition leads to the release of TNFα, IL-1β and IL-6 from neutrophils and macrophages. In this model, C5aR and FcγR cooperate to amplify injury, and genetic or pharmacological inhibition of C5aR results in partial protection (Baumann et al., 2000; Hopken et al., 1997). Tace^{ΔMC} mice showed markedly reduced levels of soluble TNFα in peritoneal lavage in response to immune complex peritonitis (FIG. 3A).

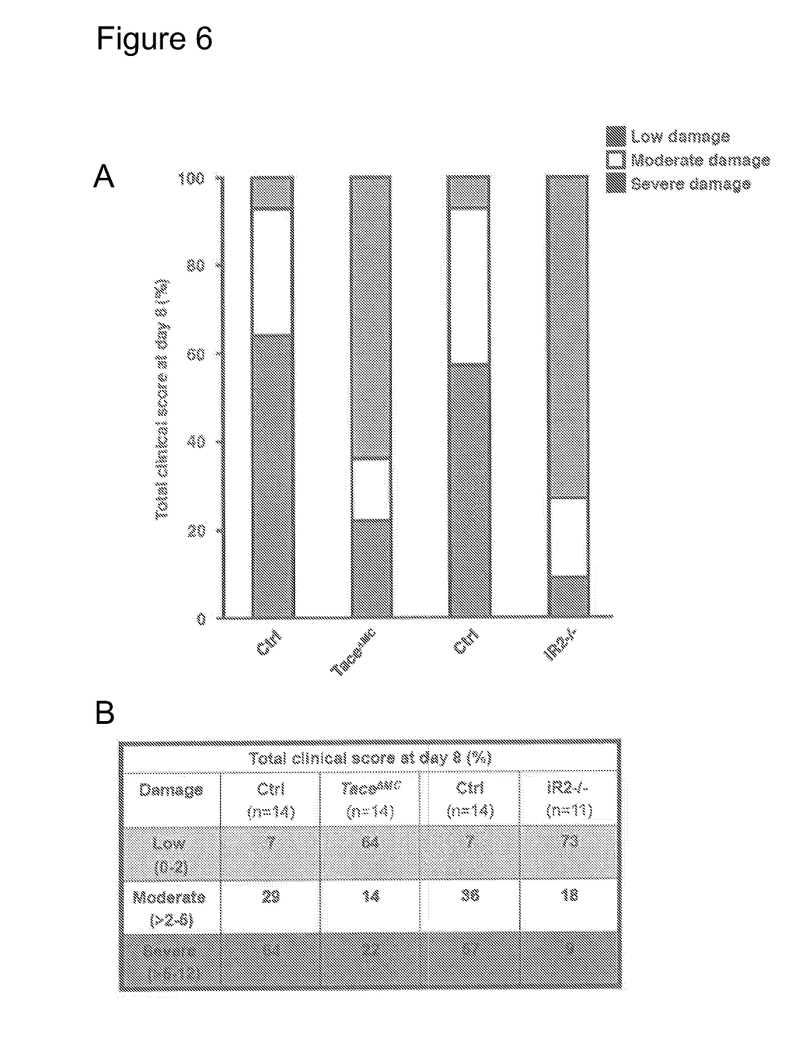

To determine the importance of activation of TACE in myeloid cells to the development of inflammatory arthritis in vivo, we next subjected Tace^{ΔMC} mice to the K/B×N passive serum transfer model, which is known to depend on TNFα, and expression of C5aR and FcγR on neutrophils and macrophages (Ji et al., 2002a; Ji et al., 2002b). Tace^{ΔMC} mice had significantly attenuated arthritis compared to control, with less joint swelling (FIG. 3B) and lower clinical scores (FIG. 3C), reflecting less synovial inflammation and cartilage erosion (FIG. 3D, E, a breakdown of the percentage of mice responding with low, moderate or severe damage is shown in FIG. 6). These results demonstrate that TACE in myeloid cells plays an important role in the development of arthritis in the K/B×N model.

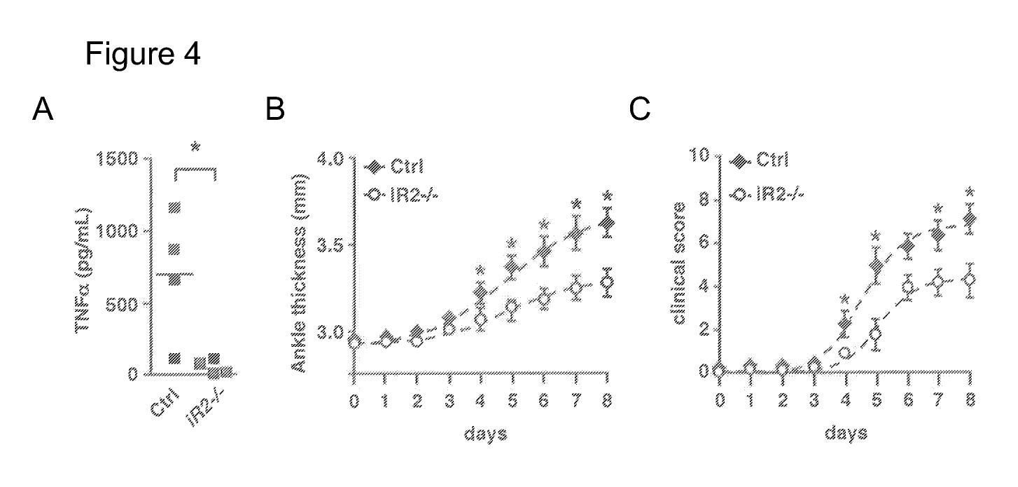

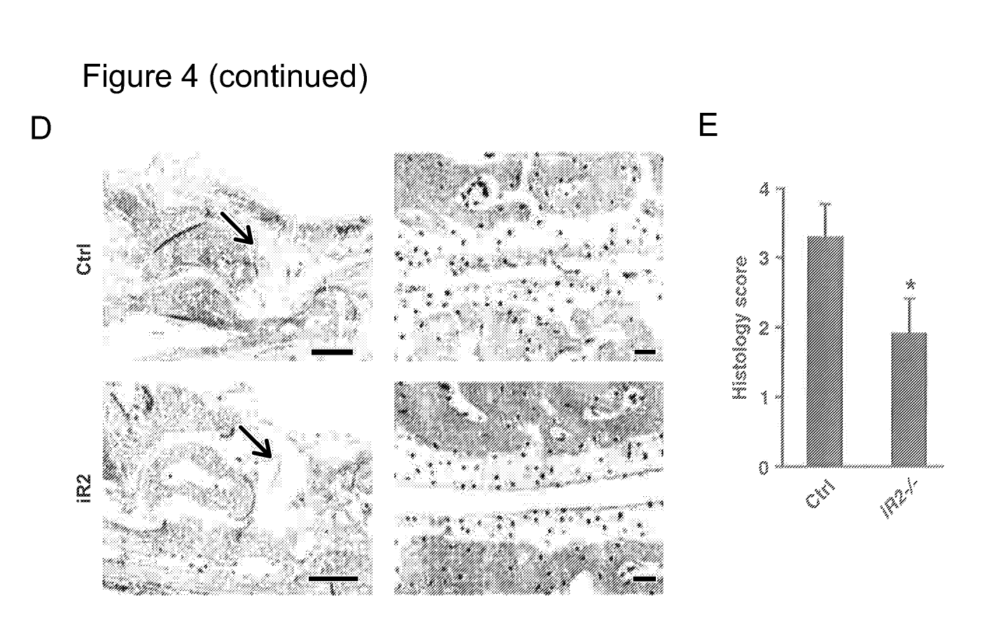

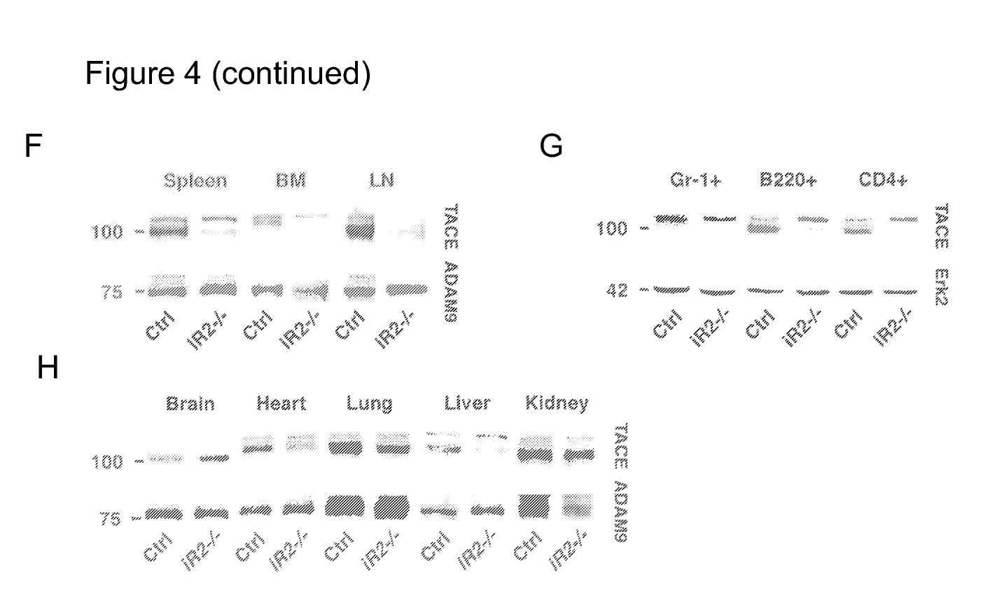

When we subjected iRhom2−/− mice to the reverse passive Arthus reaction, we found a significantly lower amount of soluble TNFα in the peritoneal lavage compared to controls (FIG. 4A). When iRhom2−/− mice were injected with K/B×N serum, we observed reduced joint swelling and histopathological features of inflammatory arthritis compared to littermate controls (FIG. 4B-E, FIG. 6), similar to the results obtained with Tace^{ΔMC} mice (FIG. 3B-E, FIG. 6). Like Tace^{ΔMC} mice, iRhom2−/− mice were partially protected from synovitis, reminiscent to observations made in Tnfα−/− mice (Ji et al., 2002b; Kitaura et al., 2004).

Systemic inactivation of TACE in mice leads to perinatal lethality (Horiuchi et al., 2007; Peschon et al., 1998), whereas iRhom2−/− mice are healthy with no spontaneous pathological phenotypes (see (Adrain et al., 2012; McIlwain et al., 2012; Siggs et al., 2012), and materials and methods).

In summary our findings have identified TACE and iRhom2 as two critical components of the pathway underlying the stimulation of TNFα release by C5a and FcγRs in myeloid cells. We show that complement C5a and FcγR rapidly activate TACE in myeloid cells, and that soluble TNFα released by TACE further enhances the expression of TNFα, IL-1β, CXCL2 and iRhom2, a crucial regulator of TACE. We also found that expression of iRhom2 is significantly increased in synovial macrophages from RA patients, and reduced by treatment with etanercept (Gordon et al., 2012). Inactivation of TACE in myeloid cells or systemic inactivation of iRhom2 protected from inflammatory arthritis and the reverse passive Arthus reaction (this study) and endotoxin shock (Horiuchi et al., 2007; McIlwain et al., 2012). Remarkably, iRhom2−/− mice are viable and fertile with no obvious pathologies, whereas mice lacking TACE die shortly after birth (Horiuchi et al., 2007; Peschon et al., 1998). This difference in phenotype can most likely be explained by the production of mature TACE in most somatic tissues of iRhom2−/− mice, which presumably allows for the normal development of these animals. Since TACE in myeloid cells is crucial for the initiation and progression of arthritis, and since iRhom2−/− mice lack mature TACE in immune cells, but not in other tissues, targeting iRhom2 may offer the unique and exciting opportunity for cell type-specific blockade of TNFα shedding. The recent description of skin abnormalities and intestinal inflammation in a patient lacking TACE (Blaydon et al., 2011) emphasizes the important opportunity provided by iRhom2 as a target to limit immune cell-specific TACE activation and avoid the systemic inactivation of TACE to treat RA and other TNFα-dependent pathologies.

### REFERENCES

Adrain, C., M. Zettl, Y. Christova, N. Taylor, and M. Freeman. 2012. Tumor necrosis factor signaling requires iRhom2 to promote trafficking and activation of TACE. Science 335:225-228.
Antoniv, T. T., and L. B. Ivashkiv. 2006. Dysregulation of interleukin-10-dependent gene expression in rheumatoid arthritis synovial macrophages. Arthritis Rheum 54:2711-2721.
Bashir, M. M., M. R. Sharma, and V. P. Werth. 2009. UVB and proinflammatory cytokines synergistically activate TNF-alpha production in keratinocytes through enhanced gene transcription. J Invest Dermatol 129:994-1001.
Baumann, U., J. Kohl, T. Tschernig, K. Schwerter-Strumpf, J. S. Verbeek, R. E. Schmidt, and J. E. Gessner. 2000. A codominant role of Fc gamma RI/III and C5aR in the reverse Arthus reaction. J Immunol 164:1065-1070.
Black, R., C. T. Rauch, C. J. Kozlosky, J. J. Peschon, J. L. Slack, M. F. Wolfson, B. J. Castner, K. L. Stocking, P. Reddy, S. Srinivasan, N. Nelson, N. Boiani, K. A. Schooley, M. Gerhart, R. Davis, J. N. Fitzner, R. S. Johnson, R. J. Paxton, C. J. March, and D. P. Cerretti. 1997. A metalloprotease disintegrin that releases tumour-necrosis factor-a from cells. Nature 385:729-733.
Blaydon, D. C., P. Biancheri, W. L. Di, V. Plagnol, R. M. Cabral, M. A. Brooke, D. A. van Heel, F. Ruschendorf, M. Toynbee, A. Walne, E. A. O'Toole, J. E. Martin, K. Lindley, T. Vulliamy, D. J. Abrams, T. T. MacDonald, J. I. Harper, and D. P. Kelsell. 2011. Inflammatory skin and bowel disease linked to ADAM17 deletion. N Engl J Med 365:1502-1508.
Burg, M., U. Martin, D. Bock, C. Rheinheimer, J. Kohl, W. Bautsch, and A. Klos. 1996. Differential regulation of the C3a and C5a receptors (CD88) by IFN-gamma and PMA in U937 cells and related myeloblastic cell lines. J Immunol 157:5574-5581.
Choe, J. Y., B. Crain, S. R. Wu, and M. Corr. 2003. Interleukin 1 receptor dependence of serum transferred arthritis can be circumvented by toll-like receptor 4 signaling. J Exp Med 197:537-542.
Durandy, A., S. V. Kaveri, T. W. Kuijpers, M. Basta, S. Miescher, J. V. Ravetch, and R. Rieben. 2009. Intravenous immunoglobulins—understanding properties and mechanisms. Clin Exp Immunol 158 Suppl 1:2-13.
Ehrengruber, M. U., T. Geiser, and D. A. Deranleau. 1994. Activation of human neutrophils by C3a and C5A. Comparison of the effects on shape changes, chemotaxis, secretion, and respiratory burst. FEBS Lett 346:181-184.
Firestein, G. S. 2003. Evolving concepts of rheumatoid arthritis. Nature 423:356-361.
Gordon, R. A., G. Grigoriev, A. Lee, G. D. Kalliolias, and L. B. Ivashkiv. 2012. The Interferon Signature and STAT1 expression in R A synovial fluid macrophages are induced by TNFα and counterregulated by synovial fluid microenvironment. Arthritis Rheumatism (in press):
Guergnon, J., M. Chaussepied, P. Sopp, R. Lizundia, M. F. Moreau, B. Blumen, D. Werling, C. J. Howard, and G. Langsley. 2003. A tumour necrosis factor alpha autocrine loop contributes to proliferation and nuclear factor-kappaB activation of Theileria parva-transformed B cells. Cell Microbiol 5:709-716.
Ha, J., H. S. Choi, Y. Lee, H. J. Kwon, Y. W. Song, and H. H. Kim. 2010. CXC chemokine ligand 2 induced by receptor activator of NF-kappa B ligand enhances osteoclastogenesis. J Immunol 184:4717-4724.
Hopken, U. E., B. Lu, N. P. Gerard, and C. Gerard. 1997. Impaired inflammatory responses in the reverse arthus reaction through genetic deletion of the C5a receptor. J Exp Med 186:749-756.
Horiuchi, K., T. Kimura, T. Miyamoto, H. Takaishi, Y. Okada, Y. Toyama, and C. P. Blobel. 2007. Cutting Edge: TNF-{alpha}-Converting Enzyme (TACE/ADAM17) Inactivation in Mouse Myeloid Cells Prevents Lethality from Endotoxin Shock. J Immunol 179:2686-2689.
Hu, X., C. Herrero, W. P. Li, T. T. Antoniv, E. Falck-Pedersen, A. E. Koch, J. M. Woods, G. K. Haines, and L. B. Ivashkiv. 2002. Sensitization of IFN-gamma Jak-STAT signaling during macrophage activation. Nat Immunol 3:859-866.
Hundhausen, C., D. Misztela, T. A. Berkhout, N. Broadway, P. Saftig, K. Reiss, D. Hartmann, F. Fahrenholz, R. Postina, V. Matthews, K. J. Kallen, S. Rose-John, and A. Ludwig. 2003. The disintegrin-like metalloproteinase ADAM10 is involved in constitutive cleavage of CX3CL1 (fractalkine) and regulates CX3CL1-mediated cell-cell adhesion. Blood 102:1186-1195.
Ji, H., K. Ohmura, U. Mahmood, D. M. Lee, F. M. Hofhuis, S. A. Boackle, K. Takahashi, V. M. Holers, M. Walport, C. Gerard, A. Ezekowitz, M. C. Carroll, M. Brenner, R. Weissleder, J. S. Verbeek, V. Duchatelle, C. Degott, C. Benoist, and D. Mathis. 2002a. Arthritis critically dependent on innate immune system players. Immunity 16:157-168.
Ji, H., A. Pettit, K. Ohmura, A. Ortiz-Lopez, V. Duchatelle, C. Degott, E. Gravallese, D. Mathis, and C. Benoist. 2002b. Critical roles for interleukin 1 and tumor necrosis factor alpha in antibody-induced arthritis. J Exp Med 196:77-85.
Kitaura, H., M. S. Sands, K. Aya, P. Zhou, T. Hirayama, B. Uthgenannt, S. Wei, S. Takeshita, D. V. Novack, M. J. Silva, Y. Abu-Amer, F. P. Ross, and S. L. Teitelbaum. 2004. Marrow stromal cells and osteoclast precursors differentially contribute to TNF-alpha-induced osteoclastogenesis in vivo. J Immunol 173:4838-4846.
Maretzky, T., W. Zhou, X. Y. Huang, and C. P. Blobel. 2011. A transforming Src mutant increases the bioavailability of EGFR ligands via stimulation of the cell-surface metalloproteinase ADAM17. Oncogene 30:611-618.
McIlwain, D. R., P. A. Lang, T. Maretzky, K. Hamada, K. Ohishi, S. K. Maney, T. Berger, A. Murthy, G. Duncan, H. C. Xu, K. S. Lang, D. Haussinger, A. Wakeham, A. Itie-Youten, R. Khokha, P. S. Ohashi, C. P. Blobel, and T. W. Mak. 2012. iRhom2 regulation of TACE controls TNF-mediated protection against Listeria and responses to LPS. Science 335:229-232.
McInnes, I. B., and G. Schett. 2011. The pathogenesis of rheumatoid arthritis. N Engl J Med 365:2205-2219.
Moss, M. L., S.-L. C. Jin, M. E. Milla, W. Burkhart, H. L. Cartner, W.-J. Chen, W. C. Clay, J. R. Didsbury, D. Hassler, C. R. Hoffman, T. A. Kost, M. H. Lambert, M. A. Lessnitzer, P. McCauley, G. McGeehan, J. Mitchell, M. Moyer, G. Pahel, W. Rocque, L. K. Overton, F. Schoenen, T. Seaton, J.-L. Su, J. Warner, D. Willard, and J. D. Becherer. 1997. Cloning of a disintegrin metalloproteinase that processes precursor tumour-recrosis factor-a. Nature 385:733-736.
Ohmura, K., A. Johnsen, A. Ortiz-Lopez, P. Desany, M. Roy, W. Besse, J. Rogus, M. Bogue, A. Puech, M. Lathrop, D. Mathis, and C. Benoist. 2005. Variation in IL-1beta gene expression is a major determinant of genetic differences in arthritis aggressivity in mice. Proc Natl Acad Sci USA 102:12489-12494.
Peschon, J. J., J. L. Slack, P. Reddy, K. L. Stocking, S. W. Sunnarborg, D. C. Lee, W. E. Russel, B. J. Castner, R. S. Johnson, J. N. Fitzner, R. W. Boyce, N. Nelson, C. J. Kozlosky, M. F. Wolfson, C. T. Rauch, D. P. Cerretti, R. J. Paxton, C. J. March, and R. A. Black. 1998. An essential role for ectodomain shedding in mammalian development. Science 282:1281-1284.
Schlöndorff, J., J. D. Becherer, and C. P. Blobel. 2000. Intracellular maturation and localization of the tumour necrosis factor alpha convertase (TACE). Biochem. J. 347 Pt 1:131-138.
Siggs, O. M., N. Xiao, Y. Wang, H. Shi, W. Tomisato, X. Li, Y. Xia, and B. Beutler. 2012. iRhom2 is required for the secretion of mouse TNF alpha. Blood
Tape, C. J., S. H. Willems, S. L. Dombernowsky, P. L. Stanley, M. Fogarasi, W. Ouwehand, J. McCafferty, and G. Murphy. 2011. Cross-domain inhibition of TACE ectodomain. Proc Natl Acad Sci USA 108:5578-5583.

What is claimed is:

1. A method for treating a subject with a Complement mediated disease or an immune complex mediated disease, comprising the step of administering to the subject an effective amount of an agent that decreases the biological activity of iRhom2.

2-10. (canceled)

11. A method for treating a subject with a Complement mediated disease or an immune complex mediated disease, comprising the step of administering to the subject an effective amount of an agent that modulates formation of a complex between iRhom2 and TACE.

12-20. (canceled)

21. A method of identifying an agent for the treatment of a disease mediated by a Complement or a disease mediated by immune complexes, comprising the steps of:
a) combining TACE, iRhom2 and a test agent under conditions suitable for forming a complex between TACE and iRhom2; and
b) assessing the quantity of complex formation between TACE and iRhom2, wherein diminished or increased complex formation between TACE and IRhom2 in the presence of the test agent than in the absence is indicative that the test agent is useful for the treatment of a disease mediated by a Complement or a disease mediated by immune complexes.

22. The method of claim 21, further comprising the steps of:
c) repeating steps a) and b) one or more times with a different test agent;
d) selecting the test agents for which the amount of complex formation between TACE and iRhom is less in the presence of the test agent or greater in the presence of the test agent than in the absence of the test agent; and
e) assaying the test agents selected in step d) in another assay for testing efficacy against a disease or disorder mediated by a Complement or immune complexes.

23. A method of identifying an agent for the treatment of a disease mediated by a Complement or a disease mediated by immune complexes, comprising the steps of:
a) combining TNF alpha, a Complement or an immune complex stimulated myeloid cells in the presence of a test agent under conditions suitable for stimulating TNF alpha release; and
b) assessing the quantity of TNF alpha release, wherein diminished TNF alpha release in the presence of the test agents than in the absence is indicative that the test agent is useful for the treatment of a disease mediated by a Complement or a disease mediated by immune complexes.

24. The method of claim 23, further comprising the steps of:
c) repeating steps a) and b) one or more time with a different test agent;
d) selecting the test agents for which the amount of TNF alpha release is diminished in the presence of the test agent than in the absence of the test agent;
e) assaying the test agents selected in step d) in another assay for testing efficacy against a disease or disorder mediated by a Complement or immune complexes.

25. (canceled)

26. The method of claim 21, wherein the Complement is C5a.

27. The method of claim 23, wherein the Complement is C5a.


SEQUENCE LISTING (1 sequence)

SEQ ID NO: 1 - protein, 52 aa, Mus sp.
GPSDKSDLSQKQPSAVVCHQDPRTCEEPASSGAHIWPDDITKWPICTEQAQS